(19) United States
(12) Patent Application Publication
(10) Pub. No.: US 2008/0262281 A1
(43) Pub. Date: Oct. 23, 2008

(54) Catalyst Used for the Oxidation of Hydrogen, and Method for the Dehydrogenation of Hydrocarbons

(75) Inventors: Christian Walsdorff, Ludwigshafen (DE); Falk Simon, Bensheim (DE); Gerald Vorberg, Speyer (DE); Gotz-Peter Schindler, Mannheim (DE); Michael Hesse, Worms (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/547,500

(22) PCT Filed: Mar. 22, 2005
(86) PCT No.: PCT/EP2005/003010

§ 371 Date: Sep. 29, 2006

(30) Foreign Application Priority Data
Mar. 29, 2004 (DE) 102004015800.2

(51) Int. Cl. C07C 5/327; B01J 23/42

(52) U.S. Cl. 585/654; 502/325; 502/334

(57) ABSTRACT

A catalyst for the oxidation of hydrogen in a process for the dehydrogenation of hydrocarbons, wherein the catalyst comprises, supported on α-aluminum oxide, from 0.01 to 0.1% by weight of platinum and from 0.01 to 0.1% by weight of tin, based on the total weight of the catalyst, a process for the oxidation of hydrogen and a process for the dehydrogenation of hydrocarbons with an integrated oxidation process using the catalyst described.

The invention relates to a catalyst for the oxidation of hydrogen in a process for the dehydrogenation of hydrocarbons, wherein the catalyst comprises, supported on α-aluminum oxide, from 0.01 to 0.1% by weight of platinum and from 0.01 to 0.1% by weight of tin, based on the total weight of the catalyst, a process for the oxidation of hydrogen and a process for the dehydrogenation of hydrocarbons with an integrated oxidation process using the catalyst specified.

The prior art describes various catalysts and processes for the oxidation of hydrogen in a dehydrogenation process.

U.S. Pat. No. 4,418,237 describes a process for the dehydrogenation of hydrocarbons with selective oxidation of the hydrogen formed in a first process stage over a dehydrogenation catalyst. The oxidation catalyst comprises noble metals of group VIII and a metal cation having an ionic radius of ≧1.35 Angstrom on a porous aluminum support having a BET surface area of from 1 to 500 m²/g. The noble metal content is in the range from 0.001 to 5% by weight.

U.S. Pat. No. 4,599,471 describes a dehydrogenation process in which an oxidation zone supplied with oxygen-rich water vapor is located between two dehydrogenation zones. The oxidation catalyst comprises a noble metal of group VIII in an amount of from 0.01 to 5% by weight and a metal or metal cation having an ionic radius of ≧1.35 Angstrom.

EP-A 826 418 describes oxidation catalysts and a process for the selective oxidation of hydrogen in the dehydrogenation of ethylbenzene to styrene. The catalysts comprise from 0.01 to 10% by weight of platinum on an aluminum oxide support, with the BET surface area of the aluminum oxide being from 0.5 to 6 m²/g and the aluminum oxide having an ammonia adsorption of not more than 5 μmol/g.

EP-A 1 229 011 describes a process for the dehydrogenation of ethylbenzene, in which an oxidation zone is integrated between two dehydrogenation stages and in whose second dehydrogenation stage a carbon dioxide generation rate of less than 2.1 based on the first stage is maintained. Oxidation catalysts used are catalysts comprising platinum, alkali metals or alkaline earth metals, tin or lead and/or metals of group 4, for example germanium.

Despite the variety of processes for the dehydrogenation of hydrocarbons having an integrated oxidation stage described in the prior art, there continues to be a need for improvement, in particular in respect of the selectivity and the economics of the integrated oxidation process.

It was accordingly an object of the invention to find a catalyst for the oxidation of hydrogen which displays a high selectivity and activity and is more economical than the catalysts of the prior art. Furthermore, an improved process for the oxidation of hydrogen, in particular integrated into a dehydrogenation process, is to be found.

This object has been achieved by means of a catalyst for the oxidation of hydrogen in a process for the dehydrogenation of hydrocarbons, wherein the catalyst comprises, supported on α-aluminum oxide, from 0.01 to 0.1% by weight of platinum and from 0.01 to 0.1% by weight of tin, based on the total weight of the catalyst.

Platinum and tin are advantageously used in a weight ratio of from 1:4 to 1:0.2, preferably in a ratio of from 1:2 to 1:0.5, in particular in a ratio of approximately 1:1.

The catalyst advantageously comprises from 0.05 to 0.09% by weight of platinum and from 0.05 to 0.09% by weight of tin, based on the total weight of the catalyst.

In addition to platinum and tin, it is possible to use, if appropriate, alkali metal compounds and/or alkaline earth metal compounds in amounts of less than 2% by weight, in particular less than 0.5% by weight. When alkali metal compounds and/or alkaline earth metal compounds are used, preference is given to alkali metal compounds, in particular sodium, potassium and/or cesium compounds.

The aluminum oxide catalyst particularly preferably contains exclusively platinum and tin. It is possible for traces of alkali metals and alkaline earth metals to be present in an order of magnitude corresponding to the compounds typically present in commercially available aluminum oxides or introduced in manufacture of the shaped bodies, for example when using magnesium stearate as tableting aid.

The catalyst support comprising α-aluminum oxide advantageously has a BET surface area of from 0.5 to 15 m²/g, preferably from 2 to 14 m²/g, in particular from 7 to 11 m²/g. Preference is given to using a shaped body as support. Preferred geometries are, for example, pellets, annular pellets, spheres, cylinders, star extrudates or cogwheel-shaped extrudates. The diameters of these geometries are advantageously from 1 to 10 mm, preferably from 2 to 8 mm, with the individual diameters being able to be distributed around the mean, abovementioned, diameter. Particular preference is given to spheres or cylinders, in particular spheres. The spheres generally have a mean diameter of from 3 to 7 mm, and it is advantageous for not more than 5% by weight of the spheres to have a diameter of less than 3 mm and not more than 5% by weight of the spheres to have a diameter greater than 7 mm.

The catalyst support preferably consists exclusively of α-aluminum oxide.

The α-aluminum oxide support can be produced by all methods known to those skilled in the art. A cylindrical shaped body is advantageously produced by mixing aluminum oxide hydrate (pseudoboehmite) powder and, if appropriate, γ-aluminum oxide powder and shaping, if appropriate with addition of auxiliaries such as graphite, magnesium stearate, potato starch or nitric acid, with addition of water in a ram extruder or preferably in a continuously operating extruder. If appropriate, the shaped bodies can also be cut to length during extrusion. The extrudates are advantageously dried at temperatures of from 100 to 180° C., generally from 400 to 800° C., preferably in a belt calciner, for from 0.5 to 5 hours. They are subsequently subjected to a final calcination, for example in a rotary tube, shaft furnace or muffle furnace, at temperatures of advantageously from 1000 to 1200° C. As an alternative, the calcination starting out from a pseudoboehmite-containing shaped body may also be carried out in a single apparatus, for example a muffle furnace, advantageously using a stepped or continuously increasing temperature profile. Mechanical properties and pore structure of the support can be influenced by the ratio of pseudoboehmite and γ-Al₂O₃. As an alternative, shaping can also be carried out by tableting, as described, for example, in EP-A 1 068 009. In the case of tableting, a preferred embodiment comprises domed annular tablets as described in U.S. Pat. No. 6,518,220.

The active components of the catalyst are generally applied by impregnation. The impregnation of the α-aluminum oxide support is carried out, for example, as described in WO 03/092887 A1. Impregnation is preferably carried out in two steps by firstly impregnating the aluminum oxide support with a solution of a platinum compound, preferably with a platinum nitrate solution, drying the catalyst and subsequently impregnating it with a solution of a tin compound, preferably with a tin(II) chloride solution, and subsequently drying and calcining it.

The catalyst of the invention advantageously has an abrasion of less than 5%. Furthermore, the catalyst of the invention advantageously has a fracture hardness of more than 10 N.

The catalyst advantageously has a shell-like profile. The bulk density is advantageously from 0.3 to 2 g/cm³, in particular from 0.6 to 1.2 g/cm³.

The catalyst of the invention can advantageously be used as oxidation catalyst. In the oxidation process of the invention, a gas mixture comprising hydrogen and hydrocarbon is reacted with an oxygen-containing gas in the presence of the oxidation catalyst of the invention. The oxygen-containing gas preferably contains at least 80% by volume of oxygen, more preferably at least 90% by volume, in particular at least 95% by volume of oxygen, in each case based on an oxygen-containing gas which is gaseous at STP, i.e. disregarding any additional dilution with water vapor. If appropriate, air can also be used. The oxidation reaction is generally carried out at a temperature of from 400 to 700 degrees celsius, in particular from 500 to 650 degrees celsius, and a pressure of from 0.3 to 10 bar, in particular from 0.4 to 1 bar. The molar ratio of oxygen to hydrogen is generally from 0.1:1 to 1:1, preferably from 0.2:1 to 0.6:1, in particular from 0.3:1 to 0.45:1. The molar ratio of hydrogen to hydrocarbons is advantageously in the range from 0.01:1 to 0.5:1, in particular from 0.1:1 to 0.3:1.

A process in which the catalyst of the invention and the oxidation process of the invention can advantageously be used is the process for the dehydrogenation of hydrocarbons, in particular alkylaromatics, particularly advantageously the dehydrogenation of ethylbenzene to styrene.

The dehydrogenation reaction is advantageously carried out in a plurality of reactors connected in series, with at least one oxidation process according to the invention being carried out between two dehydrogenation reactors or being integrated into at least one dehydrogenation reactor.

Preference is given to an assembly of three dehydrogenation reactors connected in series, with the oxidation process of the invention being integrated into the second reactor in the flow direction and, if appropriate, the third dehydrogenation reactor in the flow direction. The volume ratio of the beds of oxidation catalyst and dehydrogenation catalyst per reactor is generally from 0.1:1 to 1:1, preferably from 0.15:1 to 0.6:1, in particular from 0.2:1 to 0.4:1.

In the case of an integrated oxidation catalyst, this is preferably located upstream of the dehydrogenation catalysts, i.e. the reaction gas in the respective reactor flows firstly through the oxidation catalysts and then through the dehydrogenation catalysts. Preference is given to using radial flow reactors in which the catalyst beds of oxidation and dehydrogenation catalysts are arranged concentrically and are, if appropriate, separated from one another by cylindrical screens. The oxidation catalyst is then used as the inner bed of the two concentric, approximately hollow-cylindrical beds.

The dehydrogenation of hydrocarbons can be carried out by all processes known to those skilled in the art. The dehydrogenation of alkylaromatics to alkenylaromatics is preferably carried out in adiabatic or isothermal processes, in particular in adiabatic processes. The reaction is generally distributed over a plurality of reactors, preferably radial flow reactors (R), connected in series. Preference is given to from two to four reactors being connected in series. A fixed bed comprising dehydrogenation catalysts is located in each reactor. The dehydrogenation catalysts are generally catalysts comprising iron oxide. These are known to those skilled in the art and are described, for example, in DE-A 101 54 718. The dehydrogenation catalysts (DC) are preferably used in the form of solid cylinders, star extrudates or cogwheel-shaped extrudates, as described, for example, in EP-A 1 027 928 or EP-A 423 694. Particular preference is given to rods (solid cylinders) having a diameter (cross section) of from about 2 to 6 mm, in particular from 2.5 to 4 mm, star extrudates having a diameter of from 3 to 5 mm or cogwheel-shaped extrudates having a diameter of from 2.5 to 4 mm.

In the dehydrogenation of ethylbenzene to styrene, ethylbenzene (EB) is typically heated together with water vapor (H₂O), advantageously in an amount of less than 15% by weight based on ethylbenzene, to temperatures of about 500° C. by means of a heat exchanger (HE) and mixed with superheated steam from a steam superheater (SSH) immediately before entering the first reactor (R₁), so that the desired inlet temperature in the first reactor is usually from 600 to 650° C. The mass ratio of water vapor (total water vapor) to ethylbenzene on entry into the bed of the dehydrogenation catalyst in the first reactor is advantageously from 0.7:1 to 2.5:1. Preference is given to employing a water vapor/ethylbenzene ratio of from 0.75:1 to 1.8:1, in particular from 0.8:1 to 1.5:1. The water vapor/ethylbenzene ratio can also increase in the direction of the sub-sequent reactor stages when the oxygen fed in is diluted with water vapor. The process is preferably operated under reduced pressure; typical reactor pressures are in the range from 300 to 1000 mbar. The liquid hourly space velocity (LHSV) based on the active volume of the beds (i.e. the volume of the beds minus any dead zones through which little or no flow occurs) comprising dehydrogenation catalyst is generally from 0.2 to 0.7/h, preferably from 0.3 to 0.5/h and in particular from 0.35 to 0.45/h. Flow through the preferably hollow-cylindrical catalyst beds (radial flow reactors) is from the inside outward.

The molar ratio of oxygen (O₂) used to hydrogen discharged from the preceding reactor (R₁) is generally from 0.1:1 to 0.6:1, preferably from 0.2:1 to 0.5:1, in particular from 0.3:1 to 0.45:1, to achieve an advantageous temperature rise of 50-150° C., in particular from 70 to 130° C., over the oxidation catalyst (OC) in the second reactor (R₂). Oxygen can be fed in in the form of air or preferably in enriched form diluted with water vapor (H₂O) in order to avoid explosive mixtures. Oxygen is preferably used in a concentration of at least 80% by volume, particularly preferably at least 90% by volume and in particular at least 95% by volume, based on an oxygen-containing gas which is gaseous at STP without taking any dilution with water vapor into account.

Before entry into the third reactor (R₃), the reaction mixture is advantageously brought back to temperatures of usually from 600 to 650° C. by means of superheated steam in a heat exchanger (HE). The pressure at the outlet of the third reactor (R₃) should preferably not be more than 700 mbar, particularly preferably not more than 600 mbar and in particular not more than 500 mbar. As an alternative, in place of the heat exchanger, a further bed of the oxidation catalyst of the invention can also be stored at the inlet of R₃ in a manner analogous to the introduction of heat at the inlet of R₂, with analogous addition of oxygen.

The proportion of carbon dioxide in the gas leaving the process (dehydrogenation gas) after substantial condensation of the water vapor and the liquid hydrocarbons is preferably not more than 20% by volume, more preferably not more than 15% by volume and in particular not more than 10% by volume.

After the third reactor, the product stream is cooled and the gaseous products and the aqueous phase are separated off and the remaining stream is separated by distillation into styrene (ST) as desired product, ethylbenzene as unreacted starting material and benzene, toluene and high boilers as by-products. After the work-up of the reaction product mixture, unreacted ethylbenzene can be recirculated.

Depending on the operating conditions, a total conversion over all three reactor stages of from about 60 to 80%, in particular from 65 to 75%, is achieved. The styrene selectivities are usually from about 95 to 98%. By-products formed are mainly toluene and benzene and also hydrogen, carbon dioxide, carbon monoxide, methane, ethane and ethene.

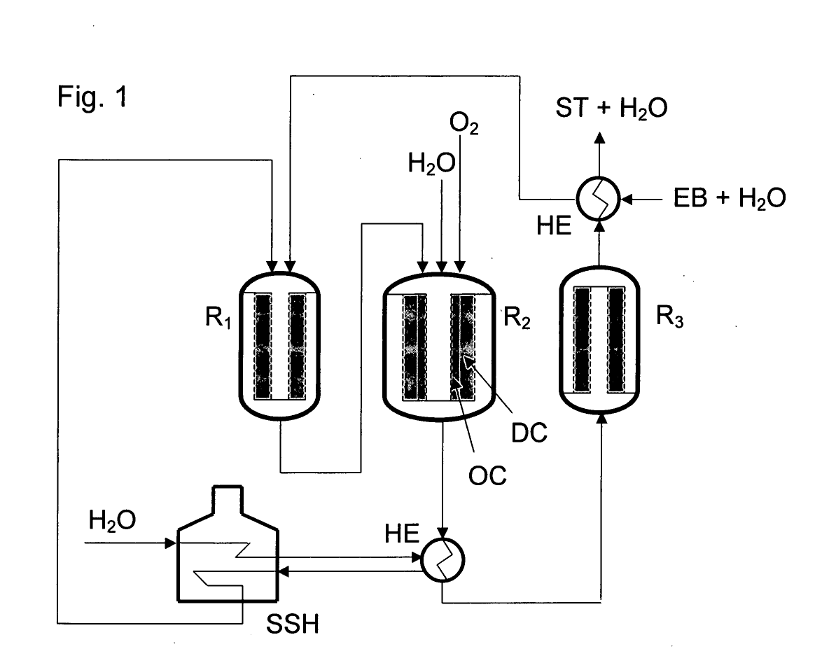

The apparatus employed in a preferred embodiment is shown schematically in FIG. 1. The number and connection of the heat exchangers are shown in simplified form; the work-up of the product mixture is not shown.

The advantage of the catalyst of the invention is its high selectivity despite a reduced mass of active catalyst. The reduction in the noble metal content thus provides a great economic advantage over the catalysts of the prior art.

### EXAMPLE

### A. Production of the Oxidation Catalyst

An α-aluminum oxide support in the form of solid cylindrical extrudates having an end face diameter of 4 mm, a water absorption of 0.38 cm³/g and a cutting hardness of 60 N was produced by extrusion of a mixture of γ-aluminum oxide and pseudoboehmite analogous to the support production example in EP 1 068 009 B1 and subsequent calcination to a BET surface area of 7 m²/g. 225 g (250 cm³) of the support were impregnated with 86 ml of a solution of 0.3134 g of platinum nitrate (57.52% platinum content). After 2 hours, the impregnated catalyst support was dried at 120° C. The catalyst was subsequently impregnated with 77 ml of a solution of 0.3427 g of tin(II) chloride dihydrate. The catalyst was then dried at 120° C. and calcined at 500° C. for 3 hours.

### B. Composition of the Oxidation Catalyst

99.84% by weight of α-aluminum oxide
0.08% by weight of platinum
0.08% by weight of tin
BET surface area of 7 m²/g

### C. Dehydrogenation of Ethylbenzene to Styrene

434 ml of catalyst as described in example 8 of DE-A 101 54 718 (using the iron oxide of example 7) in the form of 3 mm extrudates were installed in each reactor of a three-stage reactor plant having three insulated (adiabatic) tube reactors connected in series. The reactors of the first and third reactor stage were each equipped with a preheater for the inflowing gases. In the second reactor, a bed of 127 ml of the oxidation catalyst was installed above the dehydrogenation catalyst and separated from it by a 10 cm long bed of inert steatite rings. The bed of oxidation catalyst was located completely in the insulated (adiabatic) region of the reactor. Air was fed via a lance into the reaction mixture coming from the first reactor stage immediately above the bed of oxidation catalyst. (Dilution of nitrogen is preferred over dilution with steam only in the experiment, but not on an industrial scale). After the plant had been run up for seven days without introduction of air at gradually increasing load, the introduction of air into the second reactor stage was commenced from the eighth day of operation. The conditions and results obtained on the 22nd day of operation are summarized in the following table:

| | |
|---|---|
| Temperature at inlet of reactor stage 1 | 611° C. |
| Temperature at inlet of reactor stage 2 | About 525° C. |
| upstream of noble metal catalyst | |
| Temperature at inlet of reactor stage 2 | 606° C. |
| downstream of noble metal catalyst | |
| Temperature at inlet of reactor stage 3 | 613° C. |
| Pressure after reactor stage 3 | 460 mbar (absolute) |
| LHSV | 0.37/h |
| Steam/ethylbenzene (EB) | 1.45 kg/kg |
| Air introduced at inlet of reactor stage 2 | 59 standard I/h |
| upstream of noble metal catalyst | |
| EB conversion after reactor stage 3 | 71.0% |
| Styrene selectivity after reactor stage 3 | 96.6% |
| CO₂ content of the dehydrogenation gas | 4.22% by volume |

1-10. (canceled)

11. A catalyst comprising: a catalyst support, platinum and tin; wherein the catalyst support consists of α-aluminum oxide, wherein the catalyst support is impregnated with the platinum and the tin, and wherein the platinum is present in an amount of 0.01 to 0.1% by weight and the tin is present in an amount of 0.01 to 0.1% by weight based on the total weight of the catalyst.

12. The catalyst according to claim 11, wherein the weight ratio of platinum to tin is 1:4 to 1:0.2.

13. The catalyst according to claim 11, wherein the platinum is present in an amount of 0.05 to 0.09% by weight and the tin is present in an amount of 0.05 to 0.09% by weight, based on the total weight of the catalyst.

14. The catalyst according to claim 12, wherein the platinum is present in an amount of 0.05 to 0.09% by weight and the tin is present in an amount of 0.05 to 0.09% by weight, based on the total weight of the catalyst.

15. The catalyst according to claim 11, wherein the BET surface area of the α-aluminum oxide is 0.5 to 15 m²/g.

16. The catalyst according to claim 12, wherein the BET surface area of the α-aluminum oxide is 0.5 to 15 m²/g.

17. The catalyst according to claim 13, wherein the BET surface area of the α-aluminum oxide is 0.5 to 15 m²/g.

18. The catalyst according to claim 14, wherein the BET surface area of the α-aluminum oxide is 0.5 to 15 m²/g.

19. The catalyst according to claim 11, wherein catalytic metals present in the catalyst consist essentially of platinum and tin.

20. The catalyst according to claim 12, wherein catalytic metals present in the catalyst consist essentially of platinum and tin.

21. The catalyst according to claim 13, wherein catalytic metals present in the catalyst consist essentially of platinum and tin.

22. The catalyst according to claim 14, wherein catalytic metals present in the catalyst consist essentially of platinum and tin.

23. The catalyst according to claim 15, wherein catalytic metals present in the catalyst consist essentially of platinum and tin.

24. A catalyst comprising: a catalyst support, platinum and tin; wherein the catalyst support consists of α-aluminum oxide having a BET surface area of 0.5 to 15 m²/g, wherein the catalyst support is impregnated with the platinum and the tin, wherein catalytic metals present in the catalyst consist essentially of platinum and tin, wherein the platinum is present in an amount of 0.05 to 0.09% by weight and the tin is present in an amount of 0.05 to 0.09% by weight based on the total weight of the catalyst, and wherein the weight ratio of platinum to tin is 1:4 to 1:0.2.

25. A process comprising:
(a) providing a gas mixture comprising hydrogen and a hydrocarbon; and
(b) reacting the gas mixture with an oxygen-containing gas in the presence of a catalyst according to claim 11.

26. The process according to claim 25, wherein the ratio of oxygen to hydrogen is 0.1:1 to 1:1.

27. A process for the dehydrogenation of hydrocarbons, wherein dehydrogenation is carried out in a plurality of reactors connected in series and at least one oxidation process according to claim 25 is carried out between two of the plurality of dehydrogenation reactors or is integrated into at least one of the plurality of dehydrogenation reactors.

28. The process according to claim 27, wherein dehydrogenation is carried out in three dehydrogenation reactors connected in series and the oxidation process is integrated into at least one of the second of the three dehydrogenation reactors and the third dehydrogenation reactor.

29. The process according to claim 27, wherein ethylbenzene is dehydrogenated to styrene.
